(19) United States
(12) Patent Application Publication
(10) Pub. No.: US 2025/0123298 A1
(43) Pub. Date: Apr. 17, 2025

(54) METHODS, SYSTEMS AND COMPOSITIONS FOR THE PREDICTION AND PREVENTION OF PARENTERAL NUTRITION ASSOCIATED CHOLESTASIS USING FECAL BIOMARKERS

(71) Applicants: University of Virginia Patent Foundation, Charlottesville, VA (US); Inova Health Care Services, Falls Church, VA (US)

(72) Inventors: Jason Aaron Papin, Charlottesville, VA (US); Sean Ryan Moore, Charlottesville, VA (US); Thomas James Moutinho, Jr., Burlington, MA (US); Suchitra Kaveri Hourigan, Bethesda, MD (US); Gabriel Hanson, Charlottesville, VA (US)

(73) Assignees: University of Virginia Patent Foundation, Charlottesville, VA (US); Inova Health Care Services, Falls Church, VA (US)

(21) Appl. No.: 18/684,561

(22) PCT Filed: Aug. 17, 2022
(86) PCT No.: PCT/US2022/040639

§ 371 Date: Feb. 16, 2024

Related U.S. Application Data
(60) Provisional application No. 63/234,095, filed on Aug. 17, 2021.

(51) Int. Cl. G01N 33/92

(52) U.S. Cl. CPC G01N 33/92; G01N 2405/08; G01N 2800/085; G01N 2800/38; G01N 2800/50

(57) ABSTRACT

Provided are biomarkers for parenteral nutrition associated cholestasis (PNAC) in subjects, especially infants receiving parenteral nutrition (PN). Using such biomarkers provide methods of diagnosing and/or assessing risk of PNAC in a subject, including screening a sample from a subject believed to be at risk for PNAC for one or more biomarkers of PNAC. The biomarkers can include one or more fecal metabolites. Such biomarkers and associated methods provide neonatal intensive care unit clinicians with an additional tool for early identification of PNAC risk. Early identification of high-risk infants would enable clinicians to confidently optimize caloric nutrition with PN for infants at low risk of developing PNAC and enable proactive mitigation with alterations to the administered PN.

### CROSS-REFERENCE TO RELATED APPLICATIONS

This application claims benefit of and priority to U.S. Provisional Patent Application Ser. No. 63/234,095, filed Aug. 17, 2021, herein incorporated by reference in its entirety.

### GRANT STATEMENT

This invention was made with government support under TR003015 awarded by the National Institutes of Health. The government has certain rights in the invention.

### TECHNICAL FIELD

Provided are methods, systems, and compositions for the prediction of parenteral nutrition associated cholestasis using fecal sphingolipids. In some aspects, the prediction of parenteral nutrition associated cholestasis using fecal sphingolipids is in neonatal subjects, and more particularly in the neonatal intensive care unit.

### BACKGROUND

Through poorly understood mechanisms, a subset of neonates receiving parenteral nutrition (PN) develop liver damage and parenteral nutrition associated cholestasis (PNAC). PNAC is diagnosed by an elevated level of direct bilirubin in the blood that can only be detected once there is decreased bile flow through the bile ducts. There are several recognized clinical risk factors, long duration of PN, low birth weight, and young gestational age (1,2). The incidence of PNAC exceeds 50% of infants born less than 1000 grams, and 85% of infants requiring PN for longer than 14 weeks (3). Liver injury can persist even after cessation of PN, representing a significant health and economic burden (4). Recently, alternative PN lipid emulsions sourced from fish (OMEGAVEN®) or a mixture of plant and fish lipids (SMOFLIPID®) have been shown to limit the progression and injury from PNAC (5,6). However, due to multiple factors including cost, insurance limitations, and the requirement for a second intravenous line, these potentially protective lipid formulations are not utilized for all infants in the NICU (7). Therefore, there is a great need for identifying safe and effective biomarkers, early in life, to predict which infants will develop PNAC in the NICU, before clinical diagnosis of the disease to aid in diagnosing disease in the NICU (8).

Stool and urine samples, although non-invasive and with minimal risk to the infant, do not currently play a role in diagnostic protocols in the NICU. Urine and stool can, in some instances, contain valuable information about physiological processes. However, stool and urine remain an untapped resource for diagnostic applications in the NICU. It is believed that there may be an unexplored association between the development of PNAC and a differential composition in the gut microbiome. The present disclosure explored whether there are detectable and meaningful changes in the stool that may predict PNAC.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided in some embodiments are methods for predicting parenteral nutrition associated cholestasis (PNAC) and/or assessing risk of PNAC in a subject, the method comprising screening a sample from a subject believed to be at risk for PNAC for one or more biomarkers of PNAC, the biomarkers comprising one or more fecal metabolites. In some embodiments, the sample is a fecal sample from the subject. In some embodiments, the one or more biomarkers comprise one or more membrane lipids present at an elevated level as compared to a healthy subject, wherein the elevated level of the one or more membrane lipids indicates a dysregulation of lipid metabolism in the liver or gastrointestinal tract of the subject. In some embodiments, the one or more biomarkers are selected from the group consisting of sphingomyelins, glycerophosphocholine (GPC) phosphatidylcholines, GPC lysophospholipids, glycerophosphoethanolamine (GPE) lyso-lipids, primary cholic bile acids, secondary cholic bile acids, Vitamin A and Carotene diols, long-chain carnitines, and combinations thereof. In some embodiments, the one or more biomarkers are selected from the group consisting of 1-linoleoyl-glycerophosphocholine (GPC) (18:2), 1-oleoyl-2-linoleoyl-GPC (18:1/18:2), 1-oleoyl-GPC (18:1), 1-palmitoyl-GPC (16:0), 1-stearoyl-GPC (18:0), 1-stearoyl-GPE (18:0), 2-stearoyl-GPE (18:0), behenoyl sphingomyelin (d18:1/22:0), lignoceroyl sphingomyelin (d18:1/24:0), palmitoyl dihydrosphingomyelin (d18:0/16:0), palmitoyl sphingomyelin (d18:1/16:0), sphingomyelin (d17:1/16:0, d18:1/15:0, d16:1/17:0), sphingomyelin (d18:0/18:0, d19:0/17:0), sphingomyelin (d18:1/14:0, d16:1/16:0), sphingomyelin (d18:1/17:0, d17:1/18:0, d19:1/16:0), sphingomyelin (d18:1/20:0, d16:1/22:0), sphingomyelin (d18:1/20:1, d18:2/20:0), sphingomyelin (d18:1/21:0, d17:1/22:0, d16:1/23:0), sphingomyelin (d18:1/22:1, d18:2/22:0, d16:1/24:1), sphingomyelin (d18:1/24:1, d18:2/24:0), sphingomyelin (d18:2/16:0, d18:1/16:1), sphingomyelin (d18:2/23:0, d18:1/23:1, d17:1/24:1), sphingomyelin (d18:2/24:1, d18:1/24:2), stearoyl sphingomyelin (d18:1/18:0), tricosanoyl sphingomyelin (d18:1/23:0), and combinations thereof. In some embodiments, the biomarker is a sphingomyelin and/or sphingomyelin metabolite. In some embodiments, the sphingomyelin and/or sphingomyelin metabolite is present in a fecal sample from the subject.

In some embodiments, the method comprises screening a fecal sample for more than two fecal metabolites simultaneously, optionally screening a fecal sample for more than five fecal metabolites simultaneously, optionally screening a fecal sample for more than ten fecal metabolites simultaneously. In some embodiments, the subject is an infant, optionally a human infant, optionally a human infant receiving parenteral nutrition. In some embodiments, the subject is of low birth weight and/or low birth percentile, optionally wherein the subject has a birthweight less that the 40th percentile-for-gestational-age and/or less than about 1.1 kg.

In some embodiments, the methods further comprise classifying a subject as high risk of developing PNAC based the detection of one or more biomarkers, and classifying a subject as low risk of developing PNAC based the absence of one or more biomarkers. In some embodiments, the method further comprises optimizing caloric nutrition with parenteral nutrition (PN) for subjects at low risk of developing PNAC, or taking proactive mitigation measures with alterations to the administered PN for subjects at high risk of developing PNAC. In some embodiments, PNAC risk is identified before elevated conjugated bilirubin levels in the blood of the subject.

Provided herein in some embodiments are biomarkers for predicting parenteral nutrition associated cholestasis (PNAC) and/or assessing risk of PNAC in a subject, the biomarker comprising one or more fecal metabolites. In some embodiments, the one or more biomarkers comprise one or more membrane lipids present at an elevated level as compared to a healthy subject, wherein the elevated level of the one or more membrane lipids indicates a dysregulation of lipid metabolism in the liver or gastrointestinal tract of the subject. In some embodiments, the one or more biomarkers are selected from the group consisting of sphingomyelins, glycerophosphocholine (GPC) phosphatidylcholines, GPC lysophospholipids, glycerophosphoethanolamine (GPE) lyso-lipids, primary cholic bile acids, secondary cholic bile acids, Vitamin A and Carotene diols, long-chain carnitines, and combinations thereof. In some embodiments, the one or more biomarkers are selected from the group consisting of 1-linoleoyl-glycerophosphocholine (GPC) (18:2), 1-oleoyl-2-linoleoyl-GPC (18:1/18:2), 1-oleoyl-GPC (18:1), 1-palmitoyl-GPC (16:0), 1-stearoyl-GPC (18:0), 1-stearoyl-GPE (18:0), 2-stearoyl-GPE (18:0), behenoyl sphingomyelin (d18:1/22:0), lignoceroyl sphingomyelin (d18:1/24:0), palmitoyl dihydrosphingomyelin (d18:0/16:0), palmitoyl sphingomyelin (d18:1/16:0), sphingomyelin (d17:1/16:0, d18:1/15:0, d16:1/17:0), sphingomyelin (d18:0/18:0, d19:0/17:0), sphingomyelin (d18:1/14:0, d16:1/16:0), sphingomyelin (d18:1/17:0, d17:1/18:0, d19:1/16:0), sphingomyelin (d18:1/20:0, d16:1/22:0), sphingomyelin (d18:1/20:1, d18:2/20:0), sphingomyelin (d18:1/21:0, d17:1/22:0, d16:1/23:0), sphingomyelin (d18:1/22:1, d18:2/22:0, d16:1/24:1), sphingomyelin (d18:1/24:1, d18:2/24:0), sphingomyelin (d18:2/16:0, d18:1/16:1), sphingomyelin (d18:2/23:0, d18:1/23:1, d17:1/24:1), sphingomyelin (d18:2/24:1, d18:1/24:2), stearoyl sphingomyelin (d18:1/18:0), tricosanoyl sphingomyelin (d18:1/23:0), and combinations thereof. In some embodiments, the biomarker is a sphingomyelin and/or sphingomyelin metabolite. In some embodiments, the biomarker is detectable in a fecal sample.

Also provided in some embodiments are methods for treating and/or preventing parenteral nutrition associated cholestasis (PNAC) in a subject, the method comprising: predicting PNAC and/or assessing risk of PNAC in a subject, the method comprising screening a sample from a subject believed to be at risk for PNAC for one or more biomarkers of PNAC, the biomarkers comprising one or more fecal metabolites; and taking proactive mitigation measures with alterations to an administered parenteral nutrition (PN) for subjects at high risk of developing PNAC. In some embodiments, the methods further comprise classifying a subject as high risk of developing PNAC based the detection of one or more biomarkers, and classifying a subject as low risk of developing PNAC based the absence of one or more biomarkers. In some embodiments, the sample is a fecal sample from the subject. In some embodiments, the one or more biomarkers comprise one or more membrane lipids present at an elevated level as compared to a healthy subject, wherein the elevated level of the one or more membrane lipids indicates a dysregulation of lipid metabolism in the liver or gastrointestinal tract of the subject. In some embodiments, the one or more biomarkers are selected from the group consisting of sphingomyelins, glycerophosphocholine (GPC) phosphatidylcholines, GPC lysophospholipids, glycerophosphoethanolamine (GPE) lyso-lipids, primary cholic bile acids, secondary cholic bile acids, Vitamin A and Carotene diols, long-chain carnitines, and combinations thereof. In some embodiments, the one or more biomarkers are selected from the group consisting of 1-linoleoyl-glycerophosphocholine (GPC) (18:2), 1-oleoyl-2-linoleoyl-GPC (18:1/18:2), 1-oleoyl-GPC (18:1), 1-palmitoyl-GPC (16:0), 1-stearoyl-GPC (18:0), 1-stearoyl-GPE (18:0), 2-stearoyl-GPE (18:0), behenoyl sphingomyelin (d18:1/22:0), lignoceroyl sphingomyelin (d18:1/24:0), palmitoyl dihydrosphingomyelin (d18:0/16:0), palmitoyl sphingomyelin (d18:1/16:0), sphingomyelin (d17:1/16:0, d18:1/15:0, d16:1/17:0), sphingomyelin (d18:0/18:0, d19:0/17:0), sphingomyelin (d18:1/14:0, d16:1/16:0), sphingomyelin (d18:1/17:0, d17:1/18:0, d19:1/16:0), sphingomyelin (d18:1/20:0, d16:1/22:0), sphingomyelin (d18:1/20:1, d18:2/20:0), sphingomyelin (d18:1/21:0, d17:1/22:0, d16:1/23:0), sphingomyelin (d18:1/22:1, d18:2/22:0, d16:1/24:1), sphingomyelin (d18:1/24:1, d18:2/24:0), sphingomyelin (d18:2/16:0, d18:1/16:1), sphingomyelin (d18:2/23:0, d18:1/23:1, d17:1/24:1), sphingomyelin (d18:2/24:1, d18:1/24:2), stearoyl sphingomyelin (d18:1/18:0), tricosanoyl sphingomyelin (d18:1/23:0), and combinations thereof. In some embodiments, the biomarker is a sphingomyelin and/or sphingomyelin metabolite. In some embodiments, the sphingomyelin and/or sphingomyelin metabolite is present in a fecal sample from the subject.

In some embodiments, the methods comprise screening a fecal sample for more than two fecal metabolites simultaneously, optionally screening a fecal sample for more than five fecal metabolites simultaneously, optionally screening a fecal sample for more than ten fecal metabolites simultaneously. In some embodiments, the subject is an infant, optionally a human infant, optionally a human infant receiving parenteral nutrition. In some embodiments, the subject is of low birth weight and/or low birth percentile, optionally wherein the subject has a birthweight less that the 40th percentile-for-gestational-age and/or less than about 1.1 kg.

Provided in some embodiments are systems, kits, or articles of manufacture suitable for use in carrying out a method as disclosed herein. Also provided in some embodiments are systems, kits, or articles of manufacture comprising a biomarker as disclosed herein.

Accordingly, it is an object of the presently disclosed subject matter to provide methods and biomarkers for predicting parenteral nutrition associated cholestasis (PNAC) and/or assessing risk of PNAC in a subject and methods for treating and/or preventing parenteral nutrition associated cholestasis (PNAC) in a subject. These and other objects are achieved in whole or in part by the presently disclosed subject matter. Other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A-IC: Clinical characteristics of infants with and without PNAC. FIG. 1A) Continuous clinical variables were tested for a statistically significant difference between the control and PNAC groups using a Wilcoxon rank sum test. Five of the six variables were determined to be statistically different (p-value <0.05). FIG. 1B) The comparison of two clinical metrics, birth weight and birth weight percentile, reveals that there are simple thresholds that classify infants in the cohort as high or low risk. Infants born above the 40th weight percentile (adjusted for gestational age) and also weigh greater than 1.1 kg at birth are at a low risk of developing PNAC compared to the rest of the infants. FIG. 1C) Although PNAC diagnosis is correlated with the amount of time an infant receives PN, two thirds of infants diagnosed with PNAC received PN for greater than 20 days.

FIGS. 2A-2B: Metabolomics data and predictive biomarker selection. FIG. 2A) There are 60 infants in this study; 19 were diagnosed with PNAC. 200 fecal samples were collected with accompanied metabolomics data. The samples from each infant are plotted on an individual plot. The clinical conjugated bilirubin threshold, used to diagnose PNAC, is displayed with a dashed gray line on each panel. There were 9 cholestatic infants for which collection of fecal samples was possible before conjugated bilirubin levels were above the diagnostic threshold of 1 mg/dL as indicated by the dashed circle. FIG. 2B) The y-axis displays the scaled intensity for each metabolite. The case study samples were plotted as dashed lines across the boxplots. Biomarkers were selected based on statistical significance (p<0.05) and consistency among the case study samples.

FIGS. 3A-3B: Random forest machine learning with five-fold cross-validation. FIG. 3A) Feature reduction random forest machine learning was performed to determine the minimal set of clinical metrics that provide the greatest predictive potential in our cohort. The optimal random forest consists of 2 clinical metrics and has an average 5-fold cross-validation overall accuracy of 57%. Birth weight percentile adjusted by gestational age and birth weight each contribute significantly to this model. FIG. 3B) When the 78 biomarkers that were identified to have predictive potential were included, it was possible to generate a set of random forest models with greater than 70% cross-validation accuracy on average. The second set of models also demonstrates that the two previously identified clinical variables maintain predictive potential when in the context of the stool biomarkers. These models utilize all of the metabolomics samples in this study and classify samples as high or low bilirubin levels, therefore they do not predict if an infant will develop PNAC from the early stool samples collected.

FIG. 4: Biomarkers with the strongest discriminatory accuracy. This analysis includes only the infants that fall outside of the low risk group identified in FIG. 1 with a birth weight percentile above 40% and birth weight above 1.1 kg. Additionally, the number of stool samples was reduced to only include the first sample for each infant. There are 12 metabolites from our complete set of 78 that demonstrate particularly accurate discriminatory potential within our cohort. These metabolites range from being 88% to 82% accurate at classifying the infants in our cohort based on only the first fecal sample that was collected for each infant. Although these accuracies are not properly validated with independent data, they demonstrate that there are several metabolites present in NICU stool samples that have predictive capabilities. All 12 of these metabolites are various types of sphingomyelin.

FIG. 5: The 12 best biomarkers show high agreement across our cohort. There is one infant in particular that contributes the majority of false negatives across all 12 metabolites. Among the infants only false positive classifications are seen when using an ensemble majority vote across the 12 metabolites. There were 4 false positives based on majority vote and 1 false negative resulting in an overall accuracy of 85%.

FIGS. 6A-6D: Microbiota composition correlated with conjugated bilirubin levels. FIG. 6A) The infants in the cohort have microbiota that are primarily dominated by Enterobacteriaceae at the family level. Infants with conjugated bilirubin levels greater than or equal to 1 mg/dL have statistically significant higher relative abundance of Enterobacteriaceae (p<0.05). FIG. 6B) There are three known genera (Proteus, Serratia, and Shigella) within the family Enterobacteriaceae that do not align with the statistical trend at the family level. FIG. 6C) However, there are several Enterobacteriaceae with unknown genera in this study. Within this unknown group the driving signal is visible for the significant difference at the family level (p<0.05). FIG. 6D) Finally, at the species level, there is one significantly different microbe, Veillonella dispar.

FIGS. 7A and 7B: Biomarkers Negatively Correlated with PNAC. The metabolites that are statistically significantly elevated in samples with an associated conjugated bilirubin level less than 1 mg/dL were identified, while all 9 case study samples have values that are below the median value of the elevated group. FIG. 7A) The values of the 9 case study samples are displayed with the blue dashed lines. Three representative metabolites are displayed from the total set found. FIG. 7B) The total set of potentially protective metabolites are displayed with their associated p-values. The 9 case study samples are not included in either group when performing the Wilcoxon rank sum test for each metabolite.

FIG. 8: Sphingomyelin biomarkers correlate with conjugated bilirubin while maintaining predictive potential. Levels of the two most promising biomarkers were compared with conjugated bilirubin in samples taken from a subset of our cohort who had samples collected both prior to and after resolution of the development of cholestasis. These metabolites demonstrate particularly high correlation with bilirubin levels, except for the notable difference of being at high abundance in the first stool sample collected from each of the infants.

### DETAILED DESCRIPTION

Infants in the NICU are an ideal patient population for expanding upon precision medicine due to their tightly regulated nutritional sources, treatment administration, and consistent monitoring. The present disclosure is based on the belief that stool and urine samples have a role to play in monitoring the health of NICU infants, a role that has previously been unrealized. In studies disclosed herein, at least one aim was to identify the stool biomarkers that provide an early indication of disease risk. It was hypothesized that metabolites present in the stool would present earlier than the current diagnostic metric, elevated serum direct bilirubin levels, thus providing early detection of infants at risk of PNAC.

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand”, “coding sequence” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Also encompassed are any and all nucleotide sequences that encode the disclosed amino acid sequences, including but not limited to those disclosed in the corresponding GENBANK® entries.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The terms “modulate” or “alter” are used interchangeably and refer to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the terms “modulate” and/or “alter” can mean “inhibit” or “suppress”, but the use of the words “modulate” and/or “alter” are not limited to this definition.

As used herein, the terms “inhibit”, “suppress”, “repress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. By way of example and not limitation, histone methyltransferases, such as G9a, can suppress transcription of a number of genes below that observed in the absence of histone methyltransferases.

The term “RNA” refers to a molecule comprising at least one ribonucleotide residue. By “ribonucleotide” is meant a nucleotide with a hydroxyl group at the 2′ position of a D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The term “transcription factor” generally refers to a protein that modulates gene expression, such as by interaction with the cis-regulatory element and/or cellular components for transcription, including RNA Polymerase, Transcription Associated Factors (TAFs), chromatin-remodeling proteins, reverse tet-responsive transcriptional activator, and any other relevant protein that impacts gene transcription.

The term “promoter” defines a region within a gene that is positioned 5′ to a coding region of a same gene and functions to direct transcription of the coding region. The promoter region includes a transcriptional start site and at least one cis-regulatory element. The term “promoter” also includes functional portions of a promoter region, wherein the functional portion is sufficient for gene transcription. To determine nucleotide sequences that are functional, the expression of a reporter gene is assayed when variably placed under the direction of a promoter region fragment.

The terms “active”, “functional” and “physiological”, as used for example in “enzymatically active”, and “physiologically accurate”, and variations thereof, refer to the states of genes, regulatory components, etc. that are reflective of the dynamic states of each as they exists naturally, or in vivo, in contrast to static or non-active states of each. Measurements, detections or screenings based on the active, functional and/or physiologically relevant states of biological indicators can be useful in elucidating a mechanism, or defining a disease state or phenotype, as it occurs naturally. This is in contrast to measurements taken based on static concentrations or quantities of a biological indicator that are not reflective of level of activity or function thereof.

As used herein, the terms “antibody” and “antibodies” refer to proteins comprising one or more polypeptides substantially encoded by immunoglobulin genes or fragments of immunoglobulin genes. The presently disclosed subject matter also includes functional equivalents of the antibodies of the presently disclosed subject matter. As used herein, the phrase “functional equivalent” as it refers to an antibody refers to a molecule that has binding characteristics that are comparable to those of a given antibody. In some embodiments, chimerized, humanized, and single chain antibodies, as well as fragments thereof, are considered functional equivalents of the corresponding antibodies upon which they are based.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using a sequence comparison algorithm or by visual inspection.

The term “biological sample”, as used herein, refers to samples obtained from a subject, including but not limited to feces, skin, hair, tissue, blood, plasma, cells, sweat, and urine.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell, tissue, sample or subject is a cell, tissue, sample or subject being examined.

As used herein, the terms “condition”, “disease condition”, “disease”, “disease state”, and “disorder” refer to physiological states in which diseased cells or tissues, or cells/tissues of interest can be targeted with the compositions and methods of the presently disclosed subject matter. In some embodiments, a disease is PNAC.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Correspondingly, “parenteral nutrition” or “PN” is the feeding of nutritional products to a subject intravenously, bypassing the usual process of eating and digestion.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, a “subject in need thereof” is a patient, animal, mammal, or human, who will benefit from the method of this presently disclosed subject matter, e.g. a subject susceptible to developing PNAC.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

Used interchangeably herein are the terms: 1) “isolate” and “select”; and 2) “detect” and “identify”.

The term “isolated”, when used in reference to compositions, compounds, metabolites and biomarkers, refers to a particular composition, compound, metabolite and biomarker of interest at least partially isolated from other types of compounds, compositions, metabolites and biomarkers, or other cellular or biological material with which it naturally occurs in the tissue or fluid of origin. A composition, compound, metabolite and biomarker is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for metabolites and biomarkers.

### II. Subjects

The subject screened, tested, or from which a sample is taken, is desirably a human subject, although it is to be understood that the principles of the disclosed subject matter indicate that the compositions and methods are effective with respect to invertebrate and to all vertebrate species, including mammals, which are intended to be included in the term “subject”. Moreover, a mammal is understood to include any mammalian species in which screening is desirable, particularly agricultural and domestic mammalian species.

The disclosed methods are particularly useful in the testing, screening and/or treatment of warm-blooded vertebrates. Thus, the presently disclosed subject matter concerns mammals and birds.

More particularly, provided herein is the testing, screening and/or treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided herein is the treatment of livestock, including, but not limited to, domesticated swine (pigs and hogs), ruminants, horses, poultry, and the like.

In some embodiments, the subject to be used in accordance with the presently disclosed subject matter is a subject in need of treatment and/or diagnosis. In some embodiments, a subject can be an infant believed to be susceptible to developing PNAC or related condition. In some embodiments, the infant is a human infant. Infants or other juveniles of a subject as disclosed herein above can be less than one year of age, less than about 6 months of age, or less than about one month of age, or other age with respect an infant or juvenile of a particular subject species as would be apparent to one of ordinary skill in the art upon a review of the instant disclosure.

### III. Discussion of Biomarkers for PNAC and Methods of Using the Same

Parenteral nutrition associated cholestasis (PNAC) in the Neonatal Intensive Care Unit (NICU) causes significant morbidity and healthcare costs. Diagnosis of PNAC requires detection of elevated serum direct bilirubin levels after liver damage has occurred. As disclosed herein, efforts were taken to identify stool biomarkers that are present before clinical diagnosis of PNAC and predict an infant's risk of developing PNAC. Briefly, using untargeted metabolomics of 200 serial stool samples from 60 infants, statistical and machine learning approaches were applied to determine the metabolic biomarkers with the greatest predictive potential for risk of developing PNAC. Stools were collected prospectively from low birth weight babies at a level IV NICU.

As discussed in more detail in the Examples, it was found that birth weight percentile, birth weight, gestational age, duration of PN, and number of antibiotic courses all show statistically significant differences in the cohort (p<0.05). Surprisingly, 78 stool biomarkers were identified with early predictive potential (p<0.05). Moreover, 12 sphingomyelin lipids within the set of biomarkers that demonstrate high predictive potential of PNAC risk were identified based on early stool samples when utilizing clinical variables that are collected at birth.

Taken together, the present disclosure demonstrates the potential for stool metabolomics to provide NICU clinicians with an additional tool for early identification of PNAC risk. Early identification of high-risk infants can in some embodiments enable clinicians to confidently optimize caloric nutrition with parenteral nutrition (PN) for infants at low risk of developing PNAC and enable proactive mitigation with alterations to the administered PN.

Thus, in some embodiments provided are methods for predicting parenteral nutrition associated cholestasis (PNAC) and/or assessing risk of PNAC in a subject. Such methods can in some aspects include screening a sample from a subject believed to be at risk for PNAC for one or more biomarkers of PNAC, the biomarkers comprising one or more fecal metabolites. In some preferred embodiments the sample is a fecal sample from the subject, with the advantage that such samples are readily obtainable, particularly from infants.

As disclosed hereinbelow in more detail, the one or more biomarkers, including those isolated from and/or identified in a sample, comprise one or more membrane lipids present at an elevated level as compared to a healthy subject, wherein the elevated level of the one or more membrane lipids indicates a dysregulation of lipid metabolism in the liver or gastrointestinal tract of the subject. Such elevated levels can comprise any relative increase in such metabolites as compared to a standard or normal level, including for example an increase of about 5%, 10%, 15%, 20%, 25%, 30%, 40%, 50%, 60%, 70%, 80%, 90%, 95%, 99% or more.

More particularly, and only by way of example, in some aspects the one or more biomarkers can include sphingomyelins, glycerophosphocholine (GPC) phosphatidylcholines, GPC lysophospholipids, glycerophosphoethanolamine (GPE) lyso-lipids, primary cholic bile acids, secondary cholic bile acids, Vitamin A and Carotene diols, and long-chain carnitines (equal to or greater than about 10 carbons). Even more particularly, and as disclosed in the tables and Examples hereinbelow, the one or more biomarkers can include 1-linoleoyl-glycerophosphocholine (GPC) (18:2), 1-oleoyl-2-linoleoyl-GPC (18:1/18:2), 1-oleoyl-GPC (18:1), 1-palmitoyl-GPC (16:0), 1-stearoyl-GPC (18:0), 1-stearoyl-GPE (18:0), 2-stearoyl-GPE (18:0), behenoyl sphingomyelin (d18:1/22:0), lignoceroyl sphingomyelin (d18:1/24:0), palmitoyl dihydrosphingomyelin (d18:0/16:0), palmitoyl sphingomyelin (d18:1/16:0), sphingomyelin (d17:1/16:0, d18:1/15:0, d16:1/17:0), sphingomyelin (d18:0/18:0, d19:0/17:0), sphingomyelin (d18:1/14:0, d16:1/16:0), sphingomyelin (d18:1/17:0, d17:1/18:0, d19:1/16:0), sphingomyelin (d18:1/20:0, d16:1/22:0), sphingomyelin (d18:1/20:1, d18:2/20:0), sphingomyelin (d18:1/21:0, d17:1/22:0, d16:1/23:0), sphingomyelin (d18:1/22:1, d18:2/22:0, d16:1/24:1), sphingomyelin (d18:1/24:1, d18:2/24:0), sphingomyelin (d18:2/16:0, d18:1/16:1), sphingomyelin (d18:2/23:0, d18:1/23:1, d17:1/24:1), sphingomyelin (d18:2/24:1, d18:1/24:2), stearoyl sphingomyelin (d18:1/18:0), tricosanoyl sphingomyelin (d18:1/23:0), and combinations thereof.

In some embodiments, the biomarkers disclosed herein and used in the methods of diagnosing and/or treating can include a sphingomyelin and/or sphingomyelin metabolite, particularly wherein the sphingomyelin and/or sphingomyelin metabolite is present in a fecal sample from the subject, and/or isolated from a fecal sample from the subject.

In the disclosed methods of screening, diagnosing and/or preventing, fecal samples can be screened or tested for more than two fecal metabolites simultaneously, optionally a plurality of fecal metabolites simultaneously, e.g. 2, 3, 4, 5, 6, 7, 8, 9, 10, 11, 12, or more simultaneously.

The presently disclosed biomarkers and associated methods can be employed in any desired subject, including human subjects, and particularly infants, optionally human infants, and most particularly human infants receiving parenteral nutrition. Such infants can be less than one year of age, less than about 6 months of age, or less than about one month of age. As discussed further herein, the subjects can be of low birth weight and/or low birth percentile. More particularly, such subjects can have birthweight less that the 40th percentile-for-gestational-age and/or less than about 1.1 kg.

In addition to the methods of screening and/or diagnosing, provided herein are corresponding methods for treating and/or preventing PNAC in a subject. Such methods of treating and/or preventing can include predicting PNAC and/or assessing risk of PNAC in a subject by screening a sample from a subject believed to be at risk for PNAC for one or more biomarkers of PNAC as disclosed herein. Upon the detection of such a biomarker indicating a subject is at risk for PNAC or possible has/is developing PNAC, proactive mitigation measures can be employed. Such mitigation measures and/or treatments can include, but are not limited to, alterations to an administered parenteral nutrition (PN) that the subject is receiving, so as to avoid or decrease the risk of developing PNAC. Thus, in some embodiments, the use of a mitigation measure, such as an effective amount of a PN, for treating and/or preventing PNAC in a subject is provided in accordance with the presently disclosed subject matter. In some embodiments, the subject is a subject screened and/or diagnosed using one or more biomarkers and/or methods as disclosed herein.

Disclosed herein, in certain embodiments, are systems, kits and/or articles of manufacture for use with one or more methods and/or biomarkers described herein. In some embodiments, described herein is a system, kit, and/or article of manufacture for predicting parenteral nutrition associated cholestasis (PNAC) and/or assessing risk of PNAC in a subject as described herein. In some embodiments, a system, kit, and/or article of manufacture in accordance with the presently disclosed subject matter includes one or more biomarkers as described herein, libraries thereof, and/or controls, apparatuses, and reagents suitable for carrying out one or more of the methods described herein and/or for using one more biomarkers as described herein. In some embodiments, the system, kit and/or article of manufacture can comprise a point-of-care diagnostic apparatus. Representative formats for a point-of-care diagnostic apparatus include a lateral flow format, chemiluminescent assay and/or any other format as would be apparent to one of ordinary skill in the art upon a review of the instant disclosure, and as discussed further below.

The disclosed systems, kits, and/or article of manufactures, including point-of-care diagnostic apparatuses, can incorporate a variety of assay formats and associated reagents to detect one or more biomarkers of interest in a sample. A wide diversity of labels is available in the art that can be employed for conducting the subject assays. In some embodiments labels are detectable by spectroscopic, photochemical, biochemical, electrochemical, immunochemical, or other chemical approach. For example, useful labels include the radioisotopes 32P, 35S, fluorescent dyes, electron-dense reagents, enzymes. A wide variety of labels suitable for labeling biological components are known and are reported extensively in both the scientific and patent literature, and are generally applicable to the presently disclosed subject matter for the labeling of biological components or biomarkers. Suitable labels include radionucleotides, enzymes, substrates, cofactors, inhibitors, fluorescent moieties, chemiluminescent moieties, bioluminescent labels, or colorimetric labels. Reagents defining assay specificity optionally include, for example, monoclonal antibodies, polyclonal antibodies, proteins, nucleic acid probes or other polymers such as affinity matrices, carbohydrates or lipids. Detection can proceed by any of a variety of known methods, including spectrophotometric or optical tracking of radioactive, fluorescent, or luminescent markers, or other methods which track a molecule based upon size, charge or affinity. A detectable moiety can be of any material having a detectable physical or chemical property. Such detectable labels have been well-developed in the field of gel electrophoresis, column chromatography, solid substrates, spectroscopic techniques, and the like, and in general, labels useful in such methods can be applied to the present invention. Thus, a label includes without limitation any composition detectable by spectroscopic, photochemical, biochemical, immunochemical, nucleic acid probe-based, electrical, optical thermal, or other chemical approach.

In some embodiments the label is coupled directly or indirectly to a molecule to be detected, e.g. a biomarker, such as a product, substrate, or enzyme, according to methods well known in the art. As indicated above, a wide variety of labels are used, with the choice of label depending on the sensitivity required, ease of conjugation of the compound, stability requirements, available instrumentation, and disposal provisions. Non-radioactive labels are often attached by indirect approach. Generally, a receptor specific to the analyte is linked to a signal generating moiety. Sometimes the analyte receptor is linked to an adaptor molecule (such as biotin or avidin) and the assay reagent set includes a binding moiety (such as a biotinylated reagent or avidin) that binds to the adaptor and to the analyte. The analyte binds to a specific receptor on the reaction site. A labeled reagent can form a sandwich complex in which the analyte is in the center. The reagent can also compete with the analyte for receptors on the reaction site or bind to vacant receptors on the reaction site not occupied by analyte. The label is either inherently detectable or bound to a signal system, such as a detectable enzyme, a fluorescent compound, a chemiluminescent compound, or a chemiluminogenic entity such as an enzyme with a luminogenic substrate. A number of ligands and anti-ligands can be used. Where a ligand has a natural anti-ligand, for example, biotin, thyroxine, digoxigenin, and cortisol, it can be used in conjunction with labeled, anti-ligands. Alternatively, any haptenic or antigenic compound can be used in combination with an antibody.

In some embodiments the label, as used in a screening assay or point-of-care kits or diagnostic system, can also be conjugated directly to signal generating compounds, for example, by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl groups, and umbelliferone. Chemiluminescent compounds include dioxetanes, acridinium esters, luciferin, and 2,3-dihydrophthalazinediones, such as luminol.

Methods of detecting labels are well known to those of skilled in the art. Thus, for example, where the label is radioactive, approaches for detection include scintillation counting or photographic films as in autoradiography. Where the label is fluorescent, it may be detected by exciting the fluorochrome with light of an appropriate wavelength and detecting the resulting fluorescence by, for example, microscopy, visual inspection, via photographic film, by the use of electronic detectors such as digital cameras, charge coupled devices (CCDs) or photomultipliers and phototubes, or other detection device. Similarly, enzymatic labels are detected by providing appropriate substrates for the enzyme and detecting the resulting reaction product Finally, simple colorimetric labels are often detected simply by observing the color associated with the label. For example, conjugated gold often appears pink, while various conjugated beads appear the color of the bead.

In some embodiments the detectable signal may be provided by luminescence sources. Luminescence is the term commonly used to refer to the emission of light from a substance for any reason other than a rise in its temperature. In general, atoms or molecules emit photons of electromagnetic energy (e.g., light) when then move from an excited state to a lower energy state (usually the ground state). If exciting cause is a photon, the luminescence process is referred to as photoluminescence. If the exciting cause is an electron, the luminescence process can be referred to as electroluminescence. More specifically, electroluminescence results from the direct injection and removal of electrons to form an electron-hole pair, and subsequent recombination of the electron-hole pair to emit a photon. Luminescence which results from a chemical reaction is usually referred to as chemiluminescence. Luminescence produced by a living organism is usually referred to as bioluminescence. If photoluminescence is the result of a spin-allowed transition (e.g., a single-singlet transition, triplet-triplet transition), the photoluminescence process is usually referred to as fluorescence. Typically, fluorescence emissions do not persist after the exciting cause is removed as a result of short-lived excited states which may rapidly relax through such spin-allowed transitions. If photoluminescence is the result of a spin-forbidden transition (e.g., a triplet-singlet transition), the photoluminescence process is usually referred to as phosphorescence. Typically, phosphorescence emissions persist long after the exciting cause is removed as a result of long-lived excited states which may relax only through such spin-forbidden transitions. A luminescent label may have any one of the above-described properties.

Suitable chemiluminescent sources include a compound which becomes electronically excited by a chemical reaction and may then emit light which serves as the detectible signal or donates energy to a fluorescent acceptor. A diverse number of families of compounds have been found to provide chemiluminescence under a variety or conditions. One family of compounds is 2,3-dihydro-1,4-phthalazinedione. A frequently used compound is luminol, which is a 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters, for example, p-nitrophenyl and a peroxide such as hydrogen peroxide, under basic conditions. Other useful chemiluminescent compounds that are also known include —N-alkyl acridinum esters and dioxetanes. Alternatively, luciferins may be used in conjunction with luciferase or lucigenins to provide bioluminescence.

The term “analyte(s)” as used herein includes without limitation metabolites, drugs, prodrugs, pharmaceutical agents, drug metabolites, expressed proteins and cell markers, antibodies, serum proteins, cholesterol and other metabolites, polysaccharides, nucleic acids, biological analytes, biomarkers (including in particular the biomarkers disclosed herein), genes, proteins, or hormones, or any combination thereof. Analytes can be combinations of polypeptides, glycoproteins, polysaccharides, lipids, and nucleic acids. Of particular interest are biomarkers associated with a particular disease or with a specific disease stage, for example PNAC as disclosed herein.

In some embodiments, the one or more biomarkers are isolated and provided as a component of a system, kit, and/or article of manufacture, such as in a solution or suspension, or provided on a substrate or other surface. In some instances, the system, kit, and/or article of manufacture further comprises samples, such as a cell sample, and suitable solutions such as buffers or media, such as a buffer or medium for a biomarker as disclosed herein. In some embodiments, the system, kit, and/or article of manufacture can include a substrate or other surface comprising one or more biomarkers as disclosed herein. In some embodiments, additional components of the system, kit, and/or article of manufacture comprise a fecal collection tool, a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The systems, kits, and/or articles of manufactures provided herein contain packaging materials. Examples of pharmaceutical packaging materials include, but are not limited to, bottles, tubes, bags, containers, and any packaging material suitable for a selected formulation and intended mode of use, such as parenteral administration. For example, the container(s) include biomarkers, control compounds, and one or more reagents for use in a method disclosed herein, including screening methods, diagnostic methods, and/or methods of treating and/or preventing as disclosed herein. In some embodiments, a system, kit, and/or article of manufacture can comprise a mitigation measure as disclosed herein, such as an effective amount of a PN, for use in treating and/or preventing a condition in a subject as disclosed herein.

The presently disclosed systems, kits and/or articles of manufacture optionally include an identifying description or label or instructions relating to its use in the methods described herein and/or herewith. For example, a system or kit typically includes labels listing contents and/or instructions for use, and package inserts with instructions for use. A set of instructions will also typically be included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific screening, diagnostic/and/or treating and/or preventing application as disclosed herein. The label also indicates directions for use of the contents, such as in the methods described herein. As would be appreciated by one of ordinary skill in the art upon a review of the instant disclosure, additional guidance for features and aspects of a system, kit, and/or article of manufacture in accordance with the presently disclosed subject matter can be found in the below Examples.

Finally, as discussed hereinabove and detailed further in the below Examples, provided herein are novel biomarkers for predicting PNAC and/or assessing risk of PNAC in a subject. The biomarkers can generally comprise one or more fecal metabolites. As discussed herein, fecal metabolites offer advantages over other sample types and diagnostic modalities. Of particular interest in some embodiments are biomarkers including a sphingomyelin and/or sphingomyelin metabolite, particularly those detectable in and/or isolatable from the feces of a subject.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-6

Sample collection and processing: Subjects were chosen from a larger cohort of ongoing neonatal microbiome studies from the Level IV NICU at Inova Fairfax Hospital, Virginia (Western IRB approval #20210065). After informed consent, preterm and term neonates less than or equal to about 5 days of age, with an anticipated MIMCU length of stay over 5 days, were recruited. Detailed maternal, pregnancy and delivery data was collected. While in the NICU infants had stool collected twice weekly where possible. Stool was frozen at −80° C. within 12 hours. To address the aim of the current study, 60 infants were selected from the larger cohort who all had received PN for greater than 5 days. Of these, all were receiving partial enteral nutrition at the time of first stool sample collection. For stool metabolomics analyses, sample selection was enriched for PNAC cases and excluded samples in which PNAC was presumed to be secondary to infection. Infants were monitored for the development of cholestasis (defined as a conjugated bilirubin greater than or equal to about 1 mg/dL¹⁶), and the cause of cholestasis was noted as assessed by the treating physicians. Clinical data was collected at baseline and with each stool collection; this included gestational age, birth weight, antibiotic use, infections and length of time on PN.

16S rRNA gene sequencing: 327 samples underwent 16S rRNA gene sequencing of the hypervariable region V4, at either the Inova Core Research Lab or Ubiome. At the Inova Core Research lab, DNA was extracted using the DNeasy PowerSoil Pro Kit (Qiagen, CA) following manufacturer's protocol and sequenced on the Miseq platform (Illumina, CA). Samples sent to Ubiome were sequenced following previously reported methods with 150 base pair paired end reads¹⁷.

Metabolomics: 200 stool samples were prepared for metabolomic analysis performed as previously described¹⁸. Briefly, frozen samples were lyophilized and then resuspended at a 50:1 (50 μL deionized water for every 1 mg of feces weight) ratio for homogenization¹⁹. The homogenates were subjected to automated biochemical extraction and analysis by liquid chromatography and high-resolution tandem mass spectrometry (LC-MS/MS) on Metabolon's Global Platform²⁰⁻²². Raw data were extracted, peak-identified, and processed by Metabolon using proprietary software^{19,23,24}. Briefly, metabolites were identified by comparison to library entries of purified standards or recurrent unknown entities. Metabolon maintains a dynamic and proprietary biochemical reference library of more than 4,500 known metabolites (based on authenticated standards) and more than 2,000 novel metabolites (without an identified chemical structure); each library entry contains the retention time/index (RI), mass to charge ratio (m/z), and spectral data (including MS/MS fragmentation). Biochemical identifications are based on three criteria: retention index within a narrow RI window of the proposed identification, accurate mass match to the library±10 ppm, and the MS/MS forward and reverse scores between the experimental data and authentic standards. The MS/MS scores are based on a comparison of the ions present in the experimental spectrum to the ions present in the library spectrum. Three types of controls were included: a pool of small portions of each experimental sample serving as a technical replicate throughout the platform run; extracted water samples (process blanks); and a cocktail of standards spiked into every analyzed sample allowing instrument performance monitoring.

Statistical analysis: The clinical metadata was analyzed for each infant enrolled in the study as well as the metabolomics data for each of the fecal samples. For clinical data, Mann Whitney U tests were performed to correlate clinical metrics with conjugated bilirubin levels. For biomarker identification, if metabolites were correlated with conjugated bilirubin levels, Mann Whitney U tests were performed followed by a multiple testing correction using the Bonferroni method to identify metabolites differentially abundant in healthy versus control groups. Distributions were confirmed to be non-normal using the Shapiro-Wilk test.

Data processing and Machine Learning: QIIME and DADA2 were utilized to process the 16S data²⁵⁻²⁷. The forward reads from the 16S data were utilized to align with the Greengenes 16s rRNA gene database to determine taxonomic calls. The metabolomics data was processed using Python, Jupyter notebooks, pandas, scipy, numpy, and seaborn. We generated the machine learning models using Scikit Learn in Python.

### Example 1

### Experimental Design and PNAC Development in Infants

This analysis included 60 infants, all of whom received PN with Intralipid® 20% (Baxter, Deerfield, Illinois, United States of America) as the lipid emulsion. All infants were also receiving some enteral nutrition at the time of their first stool sample analyzed. 19/60 (32%) of infants developed PNAC during the study. See Table 1 for clinical and demographic characteristics of the cohort stratified by whether the infant developed PNAC. The cholestasis of these infants was not attributed to any other cause. In total, 391 stool samples were collected over the course of the study. Using these samples, 16S ribosomal RNA gene sequencing (n=327) was performed to characterize the intestinal microbiome, and liquid chromatography-mass spectrometry (LC-MS) (n=200) was conducted to measure the stool metabolome.

TABLE 1
Demographic and clinical features of the cohort stratified by
whether the infant developed PNAC

| Demographics | | No |
|---|---|---|
| and clinical characteristics | PNAC (n = 19) | PNAC (n = 41) |
| Sex: Male | 11/19 | 25/41 |
| Ethnicity: | | |
| Hispanic or Latino | 8/19 | 2/41 |
| Non-Hispanic or Non-Latino | 9/19 | 35/41 |
| Unknown | 2/19 | 14/41 |
| Race: | | |
| Black of African American | 4/19 | 6/41 |
| White or Caucasian | 6/19 | 23/41 |
| American Indian or Alaskan Native | 0/19 | 0/41 |
| Asian | 1/19 | 8/41 |
| Native Hawaiian or Pacific Islander | 0/19 | 0/41 |
| More than One Race | 3/19 | 0/41 |
| Other | 2/19 | 2/41 |
| Unknown | 3/19 | 2/41 |
| Mean gestational age in weeks (range) | 29 (23-39) | 30 (24-39) |
| Gestational age: | | |
| Extremely preterm (<27 6/7 weeks) | 10/19 | 10/41 |
| Early preterm (28 0/7-33 6/7 weeks) | 5/19 | 21/41 |
| Late preterm (34 0/7-36 6/7 weeks) | 3/19 | 6/41 |
| Early term (37 0/7-38 6/7 weeks) | 0/19 | 2/41 |
| Full term (39 0/7-40 6/7 weeks) | 4/19 | 2/41 |
| Mean birth weight in kg (range) | 1.11 | 1.61 |
| | (0.38-3.26) | (0.51-3.59) |
| Intrauterine growth restriction | 3/19 | 3/41 |
| Birth weight percentile (range) | 30.2 (0.4-97) | 50.3 (0.3-97) |
| Mode of delivery: vaginal delivery | 3/19 | 3/41 |
| Major comorbidities: | | |
| Necrotizing Enterocolitis | 3/19 | 2/41 |
| Intestinal atresia | 1/19 | 0/41 |
| Gastroschisis | 0/19 | 1/41 |
| Omphalocele | 0/19 | 0/41 |
| Systemic antibiotic use during | 17/19 | 28/41 |
| NICU course | | |
| Antibiotics immediately after birth | 17/19 | 26/41 |
| Mean length of stay in the NICU | 91 (29-151) | 60.4 (15-168) |
| in days (range) | | |
| Mean days on any TPN (range) | 29 (11-53) | 16 (6-34) |

### Example 2

### Clinical Metrics can Identify an at Risk Population of Infants on TPN

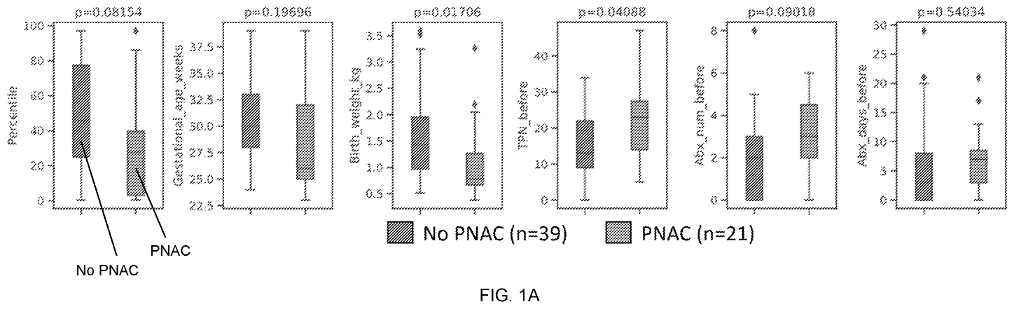

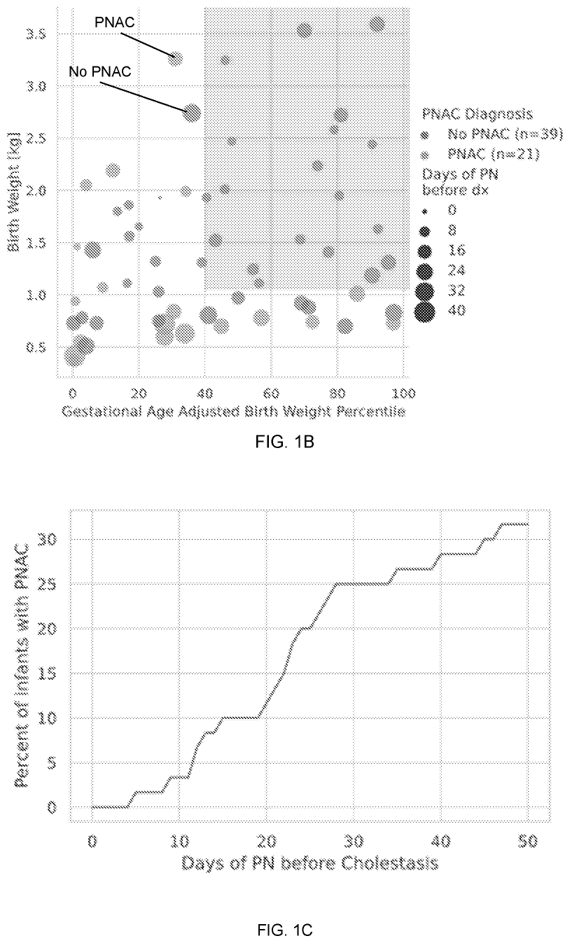

Relevant clinical data was collected for all infants enrolled in this study, including but not limited to their gestational age at birth, birth weight, number of days of PN before developing PNAC, antibiotic use, and calculated metrics such as birth weight percentile adjusted for gestational age (Table 1). Among the clinical metrics measured, only birth weight and the number of days of PN before occurrence of PNAC were statistically different between the disease and control groups (FIG. 1A; p<0.05). It was found that infants with a birth weight percentile greater than 40% and a birth weight greater than 1.1 kilograms were less likely to develop PNAC. These clinical criteria are consistent with known clinical risk factors (FIG. 1B). Since length of PN administration is associated with the development of PNAC, the relationship was investigated further in the cohort to better understand how PNAC is impacted by duration of PN. A positive correlation between PNAC diagnosis and the amount of time an infant receives PN before diagnosis (p<0.05) was observed. Two thirds of the infants diagnosed with PNAC received PN for longer than 20 days. However, the length of time on PN is limited as a very early clinical predictor of the risk of development of PNAC, as it is not concretely known until later in the clinical course although certain clinical scenarios predict longer length of PN.

### Example 3

Bacterial Taxa Correlate with PNAC, but Lack Predictive Potential in this Cohort

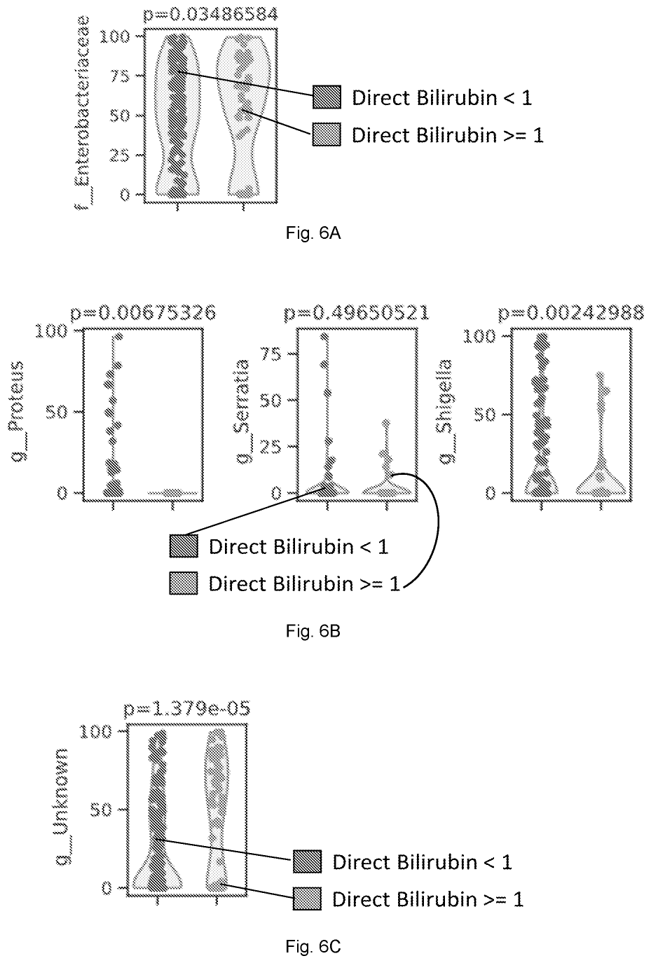

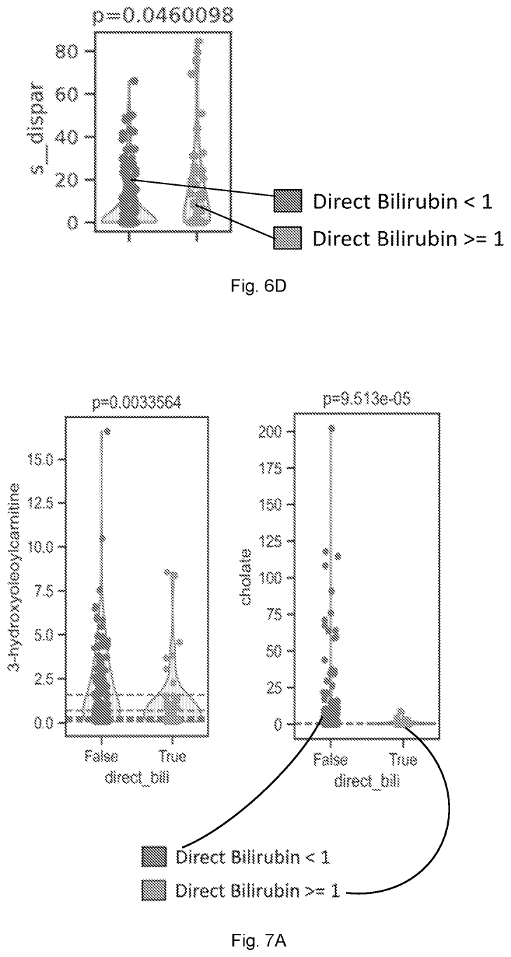

Within this cohort of NICU infants, several microbial taxa that were statistically different between disease and control groups (p<0.05) were identified. Among these taxa, the Enterobacteriaceae were present at greater abundance in stool samples collected from infants with serum conjugated bilirubin levels >=1 mg/dL (FIG. 6A). However, the three known genera within the Enterobacteriaceae family all demonstrate an opposite trend, with high abundance in the control group (FIG. 6B). The unknown genera within the Enterobacteriaceae family showed a higher abundance in samples from infants with PNAC (FIG. 6C). Thus, more research is needed to identify the primary Enterobacteriaceae genera present at elevated abundance in infants with PNAC. Finally, at the species level, Veillonella dispar was at an elevated relative abundance in infants with PNAC (FIG. 6D). It is important to note that none of these microbes showed strong associations for determining infants at risk of developing PNAC; all are simply correlated with elevated levels of conjugated bilirubin.

### Example 4

### The Stool Metabolome Contains Valuable Biomarkers for Infants at Risk of PNAC

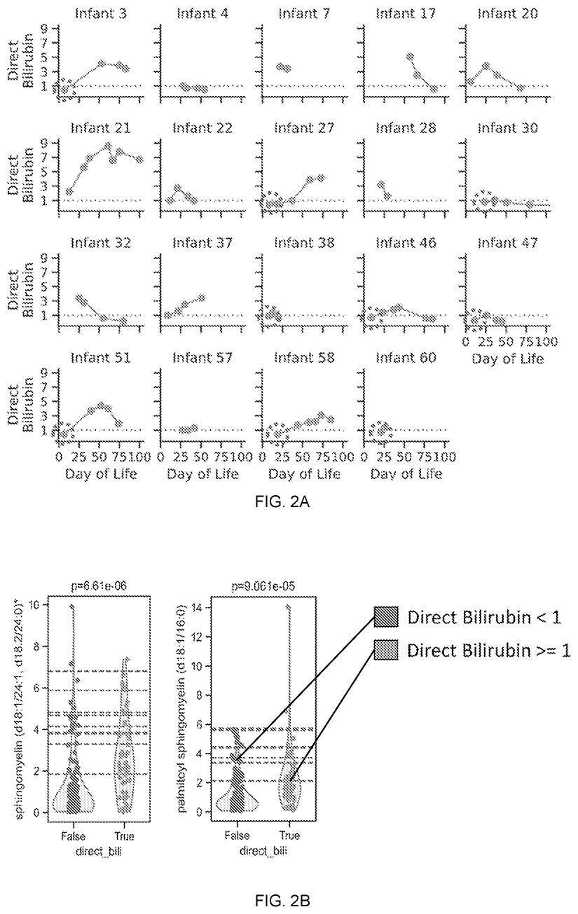

Among the 19/60 infants in the cohort who developed PNAC (FIG. 2A), there were nine for whom stool samples were collected from before a diagnosis of PNAC. The interval between fecal metabolomic measurements and the initial detection of PNAC varied based on timing of sample collection. The timing of stool sample measurements relative to the detection of PNAC in 19 patients is shown in FIG. 2A. In the 9 children (indicated by the dotted circles in FIG. 2A) from whom one or more fecal samples was collected before PNAC was diagnosed, the interval between measured elevations in fecal sphingomyelins and the detection of PNAC ranged from 5 to 46 days, with a mean and median of 20.6 and 15 days, respectively. These early samples of stools from children who went on to develop PNAC were utilized as a filter to provide a glimpse into metabolites with the greatest association for the risk of developing PNAC prior to disease progression. First, over 100 metabolites were identified with differing abundance between the disease group and the control group. For each candidate metabolite identified, the scaled quantity of that metabolite in each of the nine case study samples was compared to the median value in all of the control samples. For metabolites found to be more abundant in PNAC samples, only those with scaled intensities in greater than 90% of the nine case study samples above the median value of the PNAC distribution (FIG. 2B) were selected. For metabolites negatively correlated with disease, PNAC biomarkers were selected by identifying those with scaled intensities in greater than 90% of the nine case study samples falling below the median value of the disease distribution. Mann-Whitney U tests were performed to assess statistical significance of each metabolite between the samples with conjugated bilirubin below the clinical threshold and above. A Bonferroni multiple comparison correction was used to control for the number of metabolites tested. This analysis resulted in the identification of a subset of metabolites with the greatest potential to be early predictors for the risk of developing PNAC (Table 2).

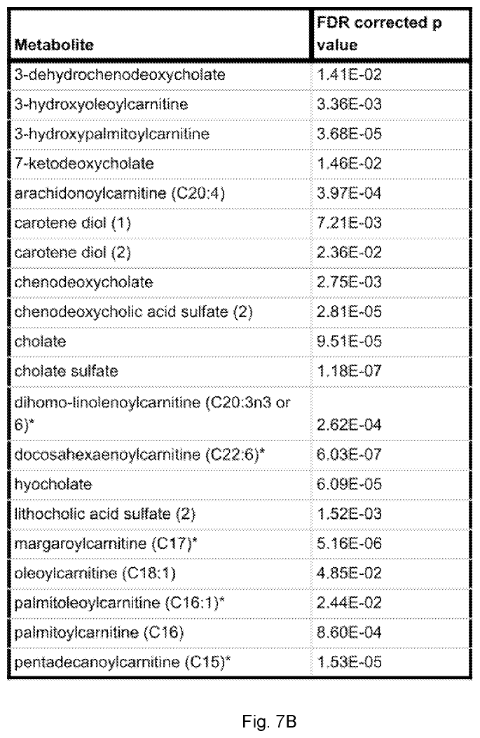

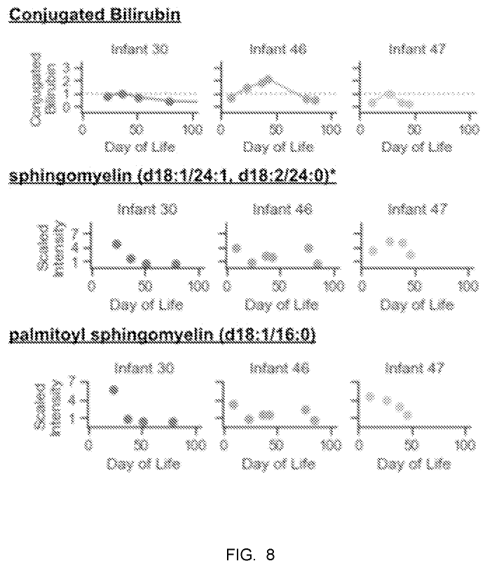

After multiple testing corrections, a total of 45 biomarkers were identified in early stool samples associated with the risk of developing PNAC. Specifically, 25 biomarkers were found to positively correlate with a subsequent rise in conjugated bilirubin levels (Table 2) and 20 metabolites negatively correlate with a subsequent increase in conjugated bilirubin levels (FIG. 7). The biomarkers elevated in PNAC samples are informative as to the pathophysiology that may be governing the disease.

TABLE 2
Biomarkers positively correlated with PNAC.
32 biomarkers that are statistically significantly elevated in samples
were identified with an associated conjugated bilirubin level
greater than or equal to 1 mg/dL, while all 9 case study samples
have values that are above the median value of the elevated group.

| | FDR corrected P |
|---|---|
| Metabolite | value |
| 1-linoleoyl-GPC (18:2) | 1.86E−05 |
| 1-oleoyl-2-linoleoyl-GPC (18:1/18:2)* | 8.63E−03 |
| 1-oleoyl-GPC (18:1) | 3.51E−05 |
| 1-palmitoyl-GPC (16:0) | 1.88E−02 |
| 1-stearoyl-GPC (18:0) | 6.01E−04 |
| 1-stearoyl-GPE (18:0) | 8.56E−07 |
| 2-stearoyl-GPE (18:0)* | 4.11E−04 |
| behenoyl sphingomyelin (d18:1/22:0)* | 1.18E−05 |
| lignoceroyl sphingomyelin (d18:1/24:0) | 6.22E−05 |
| palmitoyl dihydrosphingomyelin (d18:0/16:0)* | 1.87E−04 |
| palmitoyl sphingomyelin (d18:1/16:0) | 9.06E−05 |
| sphingomyelin (d17:1/16:0, d18:1/15:0, | 1.24E−05 |
| d16:1/17:0)* | |
| sphingomyelin (d18:0/18:0, d19:0/17:0)* | 6.09E−04 |
| sphingomyelin (d18:1/14:0, d16:1/16:0)* | 5.02E−07 |
| sphingomyelin (d18:1/17:0, d17:1/18:0, | 1.29E−05 |
| d19:1/16:0) | |
| sphingomyelin (d18:1/20:0, d16:1/22:0)* | 4.93E−06 |
| sphingomyelin (d18:1/20:1, d18:2/20:0)* | 5.71E−07 |
| sphingomyelin (d18:1/21:0, d17:1/22:0, | 7.77E−07 |
| d16:1/23:0)* | |
| sphingomyelin (d18:1/22:1, d18:2/22:0, | 8.12E−07 |
| d16:1/24:1)* | |
| sphingomyelin (d18:1/24:1, d18:2/24:0)* | 6.61E−06 |
| sphingomyelin (d18:2/16:0, d18:1/16:1)* | 1.14E−03 |
| sphingomyelin (d18:2/23:0, d18:1/23:1, | 5.74E−05 |
| d17:1/24:1)* | |
| sphingomyelin (d18:2/24:1, d18:1/24:2)* | 1.77E−06 |
| stearoyl sphingomyelin (d18:1/18:0) | 3.43E−07 |
| tricosanoyl sphingomyelin (d18:1/23:0)* | 1.73E−05 |

Individual biomarkers are quite useful, however a comparison of metabolites based on general classifications provides a measure of uniqueness for each biomarker. For example, 1000 known metabolites were identified across all samples and a total of 19 sphingomyelin metabolites. It was determined that 18 of 19 known sphingomyelin metabolites were biomarkers for elevated serum conjugated bilirubin levels. Separately, PNAC biomarkers were classified into seven additional groups (Table 3). Several other classifications of membrane lipids present at greater abundance in PNAC samples indicate a general dysregulation of lipid metabolism in the liver or GI tract. Several primary and secondary bile acids were reduced in PNAC samples, validating well-known pathophysiology. Long-chain carnitines were also reduced in PNAC samples.

TABLE 3
Biomarker classifications.
Each biomarker was classified into 10 groups. Among all of the
sphingomyelin molecules in the known set of metabolites
detected, we identified all of them to have biomarker
potential for early prediction of PNAC.

| | | | | | Positively |
|---|---|---|---|---|---|
| | | | | | or |
| | | | | | Negatively |
| | | | | Percent of | correlated |
| | | | Total | Biomarker | with |
| | | Number | in | Group | conjugated |
| | Biomarker | of Bio- | Data- | Statistically | bilirubin |
| | Classification | markers | base | Significant | levels |
| 1 | Sphingomyelins | 18 | 19 | 95% | positive |
| 2 | GPC | 1 | 13 | 8% | positive |
| | Phosphatidylcholines | | | | |
| 3 | GPC | 4 | 6 | 66% | positive |
| | Lysophospholipids | | | | |
| 4 | GPE Lyso-lipids | 2 | 8 | 25% | positive |
| 5 | Primary Cholic Bile | 4 | 17 | 24% | negative |
| | Acids | | | | |
| 6 | Secondary Cholic | 4 | 32 | 13% | negative |
| | Bile Acids | | | | |
| 7 | Vitamin A & | 2 | 4 | 50% | negative |
| | Carotene diols | | | | |
| 8 | Long-chain Carnitines | 10 | 28 | 36% | negative |
| | (>=10 carbons) | | | | |
| | Total statistically | 45 | — | — | — |
| | significant biomarkers: | | | | |
| | Total identified | — | 1000 | — | — |
| | metabolites in dataset: | | | | |

Although all of the identified biomarkers herein can in some embodiments be a valuable diagnostic tool, the sphingomyelin biomarkers presented in Table 2, Table 3, and FIG. 7 can be directly applicable to the clinical setting. By way of example and not limitation, they can predict the development of PNAC before clinical markers of cholestasis rise, i.e., diagnosing PNAC after detecting elevations in serum conjugated bilirubin levels.

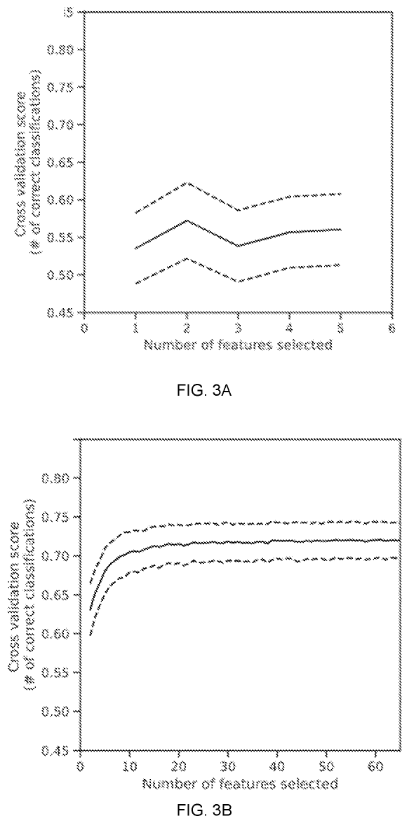

Birthweight and birthweight percentile (adjusted for gestational age) were the most important predictors of infants at risk of developing PNAC (FIG. 3A). When including metabolic biomarkers, greater than 70% overall predictive accuracy was achieved, with 5-fold cross-validation when classifying samples as control or PNAC (FIG. 3B). Five of the top six biomarkers determined to be the most predictive were sphingomyelins, as were 16 of the top 20 predictive biomarkers. Birthweight percentile and birthweight again demonstrate their predictive utility while accounting for the metabolic biomarkers. Other important biomarkers in the random forest models include several long-chain carnitines and bile acids.

### Example 5

### A Small Set of Biomarkers and Clinical Variables Provide Early Indication of Infants at Risk of PNAC

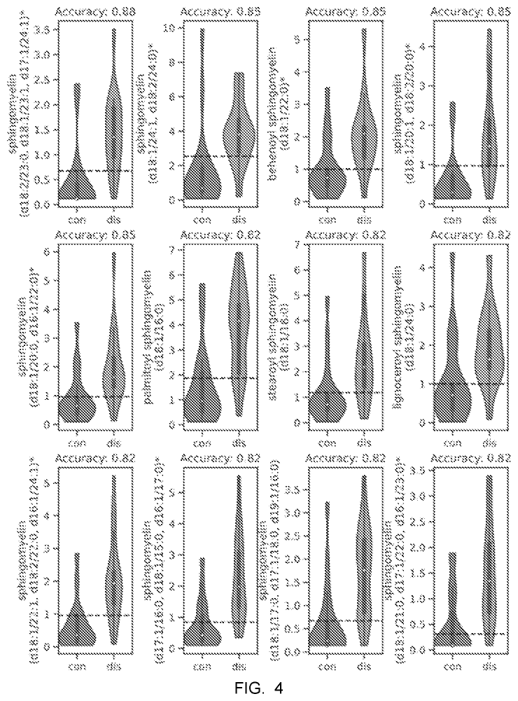

With predictive potential demonstrated using 5-fold cross-validation with a set of random forest machine learning models, the classification accuracy within the cohort was calculated using simple criteria that would be possible to implement in the clinic. The ideal implementation of these biomarkers in the clinic would be a simple point-of-care diagnostic that provides neonatologists with additional information regarding an infant's gut health. This ideal scenario requires basic thresholds to be applied to a small set of metabolites present in the stool in the first weeks of life recorded at birth which provide valuable PNAC risk stratification. In this cohort, infants with birthweights greater that the 40th percentile-for-gestational-age and greater than 1.1 kg were at significantly lower risk for developing PNAC. The other 41 infants who did not meet these cutoffs were assigned in a high-risk group to determine which fecal biomarkers provided the most robust discriminatory accuracy for the development of PNAC (FIG. 4). It was found that the 12 best biomarkers, selected from a complete set of 78, were all sphingomyelin metabolites. Each of these metabolites provided a classification accuracy of the infants in this study between 82-88%, superior to the 66% classification accuracy provided by anthropometrics alone.

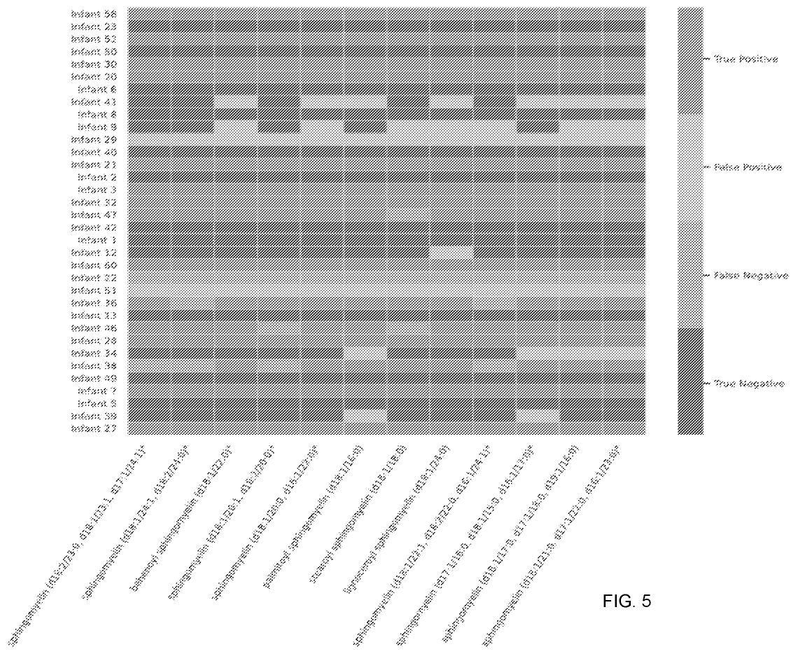

Although the 12 metabolites individually discriminated between disease and control groups in the cohort, it was important to not overfit this model and apply a more robust strategy focused on future implementation in the NICU. To improve the robustness of the calculations, the accuracy of classification when using all 12 metabolites simultaneously was assessed using an ensemble approach. For this, the first available stool samples from infants meeting the following criteria were analyzed: 1) high risk by clinical metrics, 2) stool samples collected within the first 3 weeks of life. For the 34 samples meeting this criteria, a majority-vote ensemble classifier for the cohort provides an overall accuracy of 85%, which demonstrates consistency in the alignment across the 12 metabolites identified as optimal candidates for a rigorous follow-up validation study (FIG. 5).

### Example 6

### Discussion of Results

In this NICU study of fecal biomarker predictors of PNAC in 200 fecal samples for 60 infants, at least one novel finding was the identification of 78 metabolites present in stool samples associated with an increased risk of an infant developing PNAC. Importantly, fecal metabolites in conjunction with early anthropometry provided greater predictive value than clinical factors alone. Notably, 12 sphingomyelin lipids demonstrated significant predictive potential in the cohort. This analysis identifies promising metabolites useful in a diagnostic test for early prediction of risk of developing PNAC. This work establishes that fecal sphingomyelins can inform precision clinical decision-making involving PN formulations and PNAC prevention.

PNAC is a common adverse outcome of lifesaving parenteral nutrition. Advancements in precision nutrition in the NICU may ultimately allow medical teams to improve nutrition plans and health outcomes. A key challenge in the NICU is the lack of access to frequent blood samples for longitudinal, predictive diagnostic testing. Infant stools, although not without logistical challenges, provide non-invasive, non-exhaustible samples with significant predictive potential in the NICU.

These results demonstrate clinical variables recorded at birth together with frequent testing for biomarkers in the stool would provide a method for identifying which infants are at risk of developing PNAC. Disclosed herein is a set of simple diagnostic criteria for classifying infants based on their expected risk level for developing PNAC. Early identification of PNAC before elevated conjugated bilirubin levels in the blood would allow NICU medical teams to take early action to limit the occurrence of liver damage in this vulnerable population. The most likely course of action would involve switching an infant at risk of PNAC to a hepatoprotective lipid emulsion such as that commercially available under the registered trademark OMEGAVEN®. This precise diagnostic plan would allow medical teams to proactively optimize for infant health outcomes, while also helping the NICU to account for other competing objectives, such as cost. Additionally, frequent monitoring of the stool may enable clinicians to confidently optimize caloric nutrition with PN for infants at low risk of developing PNAC, which is a known enhancer of health outcomes in the NICU.

Sphingomyelin in the stool was the most predictive fecal metabolomic signature for the risk of PNAC. This lipid plays a role in inflammatory signaling in the GI tract, tight junction maintenance, and the metabolism of nutrients present in the GI tract (28,29). The diagnostic potential that resides in biological samples that are currently treated as waste in the NICU is immense. These results demonstrate that stool samples contain measurable biomarkers that are predictive of disease. In the NICU there is a constant need for more information to help treat and take care of premature infants. Stool and urine represent two additional sources of valuable information that have previously been out of reach due to the complexity of identifying effective biomarkers for disease. However, with the advent of advanced metabolomics and systems biology, there is a new opportunity to advance diagnostic procedures in the NICU past blood tests and monitoring of vitals. PNAC is only one of many devastating diseases in the NICU that may be mitigated by early identification of infants at risk through the utilization of stool samples.

This is the first study to investigate metabolite predictors of the risk of developing PNAC. Accurate classification within the current cohort, together with high correlation of fecal sphingomyelins with serum direct bilirubin levels over time, provides support that these biomarkers for prediction of infants at risk of developing PNAC.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Hsieh M-H, Pai W, Tseng H-I, Yang S-N, Lu C-C, Chen H-L. Parenteral Nutrition-associated Cholestasis in Premature Babies: Risk Factors and Predictors. Pediatrics & Neonatology. 2009 Oct. 1; 50 (5): 202-7.
2. Alkharfy T M, Ba-Abbad R, Hadi A, Sobaih B H, AlFaleh K M. Total parenteral nutrition-associated cholestasis and risk factors in preterm infants. Saudi J Gastroenterol. 2014; 20 (5): 293-6.
3. Christensen R D, Henry E, Wiedmeier S E, Burnett J, Lambert D K. Identifying patients, on the first day of life, at high-risk of developing parenteral nutrition-associated liver disease. J Perinatol. 2007 May; 27 (5): 284-90.
4. Mangalat N, Bell C, Graves A, Imseis E M. Natural history of conjugated bilirubin trajectory in neonates following parenteral nutrition cessation. BMC Pediatrics. 2014 Dec. 10; 14 (1): 298.
5. Park H W, Lee N M, Kim J H, Kim K S, Kim S-N. Parenteral Fish Oil-Containing Lipid Emulsions May Reverse Parenteral Nutrition-Associated Cholestasis in Neonates: A Systematic Review and Meta-Analysis. The Journal of Nutrition. 2015 Feb. 1; 145 (2): 277-83.
6. Kasirer Y, Bin-Nun A, Raveh A, Schorrs I, Mimouni F B, Hammerman C. SMOFlipid Protects Preterm Neonates against Perinatal Nutrition-Associated Cholestasis. Am J Perinatol. 2019 November; 36 (13): 1382-6.
7. Casson C, Nguyen V, Nayak P, Channabasappa N, Berris K, Panczuk J, et al. A Comparison of Smoflipid® and Intralipid® in the Early Management of Infants with Intestinal Failure. J Pediatr Surg. 2020 January; 55 (1): 153-7.
8. Zhgun E S, Ilina E N. Fecal Metabolites As Non-Invasive Biomarkers of Gut Diseases. Acta Naturae. 2020 Apr. 1; 12 (2): 4-14.
9. Bouatra S, Aziat F, Mandal R, Guo A C, Wilson M R, Knox C, et al. The Human Urine Metabolome. PLOS ONE. 2013 Sep. 4; 8 (9): e73076.
10. Cahova M, Bratova M, Wohl P. Parenteral Nutrition-Associated Liver Disease: The Role of the Gut Microbiota. Nutrients. 2017 Sep. 7; 9 (9): 987.
11. Lee W S, Sokol R J. Intestinal Microbiota, Lipids, and the Pathogenesis of Intestinal Failure Associated Liver Disease. The Journal of Pediatrics. 2015 Sep. 1; 167 (3): 519-26.
12. Hourigan S K, Moutinho T J, Berenz A, Papin J, Guha P, Bangiolo L, et al. Gram-negative microbiota blooms in premature twins discordant for parenteral nutrition associated cholestasis. J Pediatr Gastroenterol Nutr. 2020 May; 70 (5): 640-4.
13. Li Y, Tang R, Leung P S C, Gershwin M E, Ma X. Bile acids and intestinal microbiota in autoimmune cholestatic liver diseases. Autoimmunity Reviews. 2017 Sep. 1; 16 (9): 885-96.
14. Bock J, Liebisch G, Schweimer J, Schmitz G, Rogler G. Exogenous sphingomyelinase causes impaired intestinal epithelial barrier function. World J Gastroenterol. 2007 Oct. 21; 13 (39): 5217-25.
15. Qi Y, Jiang C, Cheng J, Krausz K W, Li T, Ferrell J M, et al. Bile acid signaling in lipid metabolism: Metabolomic and lipidomic analysis of lipid and bile acid markers linked to anti-obesity and anti-diabetes in mice. Biochim Biophys Acta. 2015 January; 1851 (1): 19-29.
16. Kurek K, Łukaszuk B, Piotrowska D M, Wiesiołek P, Chabowska A M, Żendzian-Piotrowska M. Metabolism, Physiological Role, and Clinical Implications of Sphingolipids in Gastrointestinal Tract. BioMed Research International. 2013 Sep. 5; 2013: e908907.
17. Soo I, Madsen K L, Tejpar Q, Sydora B C, Sherbaniuk R, Cinque B, et al. VSL #3 probiotic upregulates intestinal mucosal alkaline sphingomyelinase and reduces inflammation. Can J Gastroenterol. 2008 March; 22 (3): 237-42.
18. Ramamoorthy S, Levy S, Mohamed M, Abdelghani A, Evans A M, Miller L A D, et al. An ambient-temperature storage and stabilization device performs comparably to flash-frozen collection for stool metabolomics in infants. BMC Microbiology. 2021 Feb. 22; 21 (1): 59.
19. Loftfield E, Vogtmann E, Sampson J N, Moore S C, Nelson H, Knight R, et al. Comparison of Collection Methods for Fecal Samples for Discovery Metabolomics in Epidemiologic Studies. Cancer Epidemiol Biomarkers Prev. 2016 Nov. 1; 25 (11): 1483-90.
20. Zierer J, Jackson M A, Kastenmüller G, Mangino M, Long T, Telenti A, et al. The fecal metabolome as a functional readout of the gut microbiome. Nat Genet. 2018 June; 50 (6): 790-5.
21. Evans A M, Bridgewater B, Liu Q, Mitchell M, Robinson R, Dai H, et al. High resolution mass spectrometry improves data quantity and quality as compared to unit mass resolution mass spectrometry in high-throughput profiling metabolomics. Metabolomics. 2014; 4 (2): 1.
22. Ford L, Kennedy A D, Goodman K D, Pappan K L, Evans A M, Miller L A D, et al. Precision of a Clinical Metabolomics Profiling Platform for Use in the Identification of Inborn Errors of Metabolism. The Journal of Applied Laboratory Medicine. 2020 Mar. 1; 5 (2): 342-56.
23. Evans A M, DeHaven C D, Barrett T, Mitchell M, Milgram E. Integrated, nontargeted ultrahigh performance liquid chromatography/electrospray ionization tandem mass spectrometry platform for the identification and relative quantification of the small-molecule complement of biological systems. Analytical chemistry. 2009; 81 (16): 6656-67.
24. DeHaven C D, Evans A M, Dai H, Lawton K A. Organization of GC/MS and LC/MS metabolomics data into chemical libraries. Journal of cheminformatics. 2010; 2 (1): 1-12.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for predicting parenteral nutrition associated cholestasis (PNAC) and/or assessing risk of PNAC in a subject, the method comprising screening a sample from a subject believed to be at risk for PNAC for one or more biomarkers of PNAC, the biomarkers comprising one or more fecal metabolites.

2. The method of claim 1, wherein the sample is a fecal sample from the subject.

3. The method of any of claims 1-2, wherein the one or more biomarkers comprise one or more membrane lipids present at an elevated level as compared to a healthy subject, wherein the elevated level of the one or more membrane lipids indicates a dysregulation of lipid metabolism in the liver or gastrointestinal tract of the subject.

4. The method of any of claims 1-3, wherein the one or more biomarkers are selected from the group consisting of sphingomyelin, glycerophosphocholine (GPC) phosphatidylcholine, GPC lysophospholipid, glycerophosphoethanolamine (GPE) lyso-lipid, primary cholic bile acid, secondary cholic bile acid, Vitamin A and Carotene diol, long-chain carnitine, and combinations thereof.

5. The method of any of claims 1-4, wherein the one or more biomarkers are selected from the group consisting of 1-linoleoyl-glycerophosphocholine (GPC) (18:2), 1-oleoyl-2-linoleoyl-GPC (18:1/18:2), 1-oleoyl-GPC (18:1), 1-palmitoyl-GPC (16:0), 1-stearoyl-GPC (18:0), 1-stearoyl-GPE (18:0), 2-stearoyl-GPE (18:0), behenoyl sphingomyelin (d18:1/22:0), lignoceroyl sphingomyelin (d18:1/24:0), palmitoyl dihydrosphingomyelin (d18:0/16:0), palmitoyl sphingomyelin (d18:1/16:0), sphingomyelin (d17:1/16:0, d18:1/15:0, d16:1/17:0), sphingomyelin (d18:0/18:0, d19:0/17:0), sphingomyelin (d18:1/14:0, d16:1/16:0), sphingomyelin (d18:1/17:0, d17:1/18:0, d19:1/16:0), sphingomyelin (d18:1/20:0, d16:1/22:0), sphingomyelin (d18:1/20:1, d18:2/20:0), sphingomyelin (d18:1/21:0, d17:1/22:0, d16:1/23:0), sphingomyelin (d18:1/22:1, d18:2/22:0, d16:1/24:1), sphingomyelin (d18:1/24:1, d18:2/24:0), sphingomyelin (d18:2/16:0, d18:1/16:1), sphingomyelin (d18:2/23:0, d18:1/23:1, d17:1/24:1), sphingomyelin (d18:2/24:1, d18:1/24:2), stearoyl sphingomyelin (d18:1/18:0), tricosanoyl sphingomyelin (d18:1/23:0), and combinations thereof.

6. The method of claim 1, wherein the biomarker is a sphingomyelin and/or sphingomyelin metabolite.

7. The method of claim 6, wherein the sphingomyelin and/or sphingomyelin metabolite is present in a fecal sample from the subject.

8. The method of any of claims 1-5, comprising screening a fecal sample for more than two fecal metabolites simultaneously, optionally screening a fecal sample for more than five fecal metabolites simultaneously, optionally screening a fecal sample for more than ten fecal metabolites simultaneously.

9. The method of any of claims 1-8, wherein the subject is an infant, optionally a human infant, optionally a human infant receiving parenteral nutrition.

10. The method of any of claims 1-9, wherein the subject is of low birth weight and/or low birth percentile, optionally wherein the subject has a birthweight less that the 40th percentile-for-gestational-age and/or less than about 1.1 kg.

11. The method of any of claims 1-10, further comprising classifying a subject as high risk of developing PNAC based the detection of one or more biomarkers, and classifying a subject as low risk of developing PNAC based the absence of one or more biomarkers.

12. The method of claim 11, further comprising optimizing caloric nutrition with parenteral nutrition (PN) for subjects at low risk of developing PNAC, or taking proactive mitigation measures with alterations to the administered PN for subjects at high risk of developing PNAC.

13. The method of claim 11, wherein PNAC risk is identified before elevated conjugated bilirubin levels in the blood of the subject.

14. A biomarker for predicting parenteral nutrition associated cholestasis (PNAC) and/or assessing risk of PNAC in a subject, the biomarker comprising one or more fecal metabolites.

15. The biomarker of claim 14, wherein the one or more biomarkers comprise one or more membrane lipids present at an elevated level as compared to a healthy subject, wherein the elevated level of the one or more membrane lipids indicates a dysregulation of lipid metabolism in the liver or gastrointestinal tract of the subject.

16. The biomarker of claim 14 or 15, wherein the one or more biomarkers are selected from the group consisting of sphingomyelin, glycerophosphocholine (GPC) phosphatidylcholine, GPC lysophospholipid, glycerophosphoethanolamine (GPE) lyso-lipid, primary cholic bile acid, secondary cholic bile acid, Vitamin A and Carotene diol, long-chain carnitine, and combinations thereof.

17. The biomarker of any of claims 14-16, wherein the one or more biomarkers are selected from the group consisting of 1-linoleoyl-glycerophosphocholine (GPC) (18:2), 1-oleoyl-2-linoleoyl-GPC (18:1/18:2), 1-oleoyl-GPC (18:1), 1-palmitoyl-GPC (16:0), 1-stearoyl-GPC (18:0), 1-stearoyl-GPE (18:0), 2-stearoyl-GPE (18:0), behenoyl sphingomyelin (d18:1/22:0), lignoceroyl sphingomyelin (d18:1/24:0), palmitoyl dihydrosphingomyelin (d18:0/16:0), palmitoyl sphingomyelin (d18:1/16:0), sphingomyelin (d17:1/16:0, d18:1/15:0, d16:1/17:0), sphingomyelin (d18:0/18:0, d19:0/17:0), sphingomyelin (d18:1/14:0, d16:1/16:0), sphingomyelin (d18:1/17:0, d17:1/18:0, d19:1/16:0), sphingomyelin (d18:1/20:0, d16:1/22:0), sphingomyelin (d18:1/20:1, d18:2/20:0), sphingomyelin (d18:1/21:0, d17:1/22:0, d16:1/23:0), sphingomyelin (d18:1/22:1, d18:2/22:0, d16:1/24:1), sphingomyelin (d18:1/24:1, d18:2/24:0), sphingomyelin (d18:2/16:0, d18:1/16:1), sphingomyelin (d18:2/23:0, d18:1/23:1, d17:1/24:1), sphingomyelin (d18:2/24:1, d18:1/24:2), stearoyl sphingomyelin (d18:1/18:0), tricosanoyl sphingomyelin (d18:1/23:0), and combinations thereof.

18. The biomarker of any of claims 14-17, wherein the biomarker is a sphingomyelin and/or sphingomyelin metabolite.

19. The biomarker of any of claims 14-18, wherein the biomarker is detectable in a fecal sample.

20. A method for treating and/or preventing parenteral nutrition associated cholestasis (PNAC) in a subject, the method comprising:
predicting PNAC and/or assessing risk of PNAC in a subject, comprising screening a sample from a subject believed to be at risk for PNAC for one or more biomarkers of PNAC, the biomarkers comprising one or more fecal metabolites; and
taking a proactive mitigation measure with an alteration to an administered parenteral nutrition (PN) for subjects at high risk of developing PNAC.

21. The method of claim 20, further comprising classifying a subject as high risk of developing PNAC based the detection of one or more biomarkers, and classifying a subject as low risk of developing PNAC based the absence of one or more biomarkers.

22. The method of claim 20 or claim 21, wherein the sample is a fecal sample from the subject.

23. The method of any of claims 20-22, wherein the one or more biomarkers comprise one or more membrane lipids present at an elevated level as compared to a healthy subject, wherein the elevated level of the one or more membrane lipids indicates a dysregulation of lipid metabolism in the liver or gastrointestinal tract of the subject.

24. The method of any of claims 20-23, wherein the one or more biomarkers are selected from the group consisting of sphingomyelin, glycerophosphocholine (GPC) phosphatidylcholine, GPC lysophospholipid, glycerophosphoethanolamine (GPE) lyso-lipid, primary cholic bile acid, secondary cholic bile acid, Vitamin A and Carotene diol, long-chain carnitine, and combinations thereof.

25. The method of any of claims 20-24, wherein the one or more biomarkers are selected from the group consisting of 1-linoleoyl-glycerophosphocholine (GPC) (18:2), 1-oleoyl-2-linoleoyl-GPC (18:1/18:2), 1-oleoyl-GPC (18:1), 1-palmitoyl-GPC (16:0), 1-stearoyl-GPC (18:0), 1-stearoyl-GPE (18:0), 2-stearoyl-GPE (18:0), behenoyl sphingomyelin (d18:1/22:0), lignoceroyl sphingomyelin (d18:1/24:0), palmitoyl dihydrosphingomyelin (d18:0/16:0), palmitoyl sphingomyelin (d18:1/16:0), sphingomyelin (d17:1/16:0, d18:1/15:0, d16:1/17:0), sphingomyelin (d18:0/18:0, d19:0/17:0), sphingomyelin (d18:1/14:0, d16:1/16:0), sphingomyelin (d18:1/17:0, d17:1/18:0, d19:1/16:0), sphingomyelin (d18:1/20:0, d16:1/22:0), sphingomyelin (d18:1/20:1, d18:2/20:0), sphingomyelin (d18:1/21:0, d17:1/22:0, d16:1/23:0), sphingomyelin (d18:1/22:1, d18:2/22:0, d16:1/24:1), sphingomyelin (d18:1/24:1, d18:2/24:0), sphingomyelin (d18:2/16:0, d18:1/16:1), sphingomyelin (d18:2/23:0, d18:1/23:1, d17:1/24:1), sphingomyelin (d18:2/24:1, d18:1/24:2), stearoyl sphingomyelin (d18:1/18:0), tricosanoyl sphingomyelin (d18:1/23:0), and combinations thereof.

26. The method of any of claims 20-25, wherein the biomarker is a sphingomyelin and/or sphingomyelin metabolite.

27. The method of claim 26, wherein the sphingomyelin and/or sphingomyelin metabolite is present in a fecal sample from the subject.

28. The method of any of claims 20-27, comprising screening a fecal sample for more than two fecal metabolites simultaneously, optionally screening a fecal sample for more than five fecal metabolites simultaneously, optionally screening a fecal sample for more than ten fecal metabolites simultaneously.

29. The method of any of claims 20-28, wherein the subject is an infant, optionally a human infant, optionally a human infant receiving parenteral nutrition.

30. The method of claim 29, wherein the subject is of low birth weight and/or low birth percentile, optionally wherein the subject has a birthweight less that the 40th percentile-for-gestational-age and/or less than about 1.1 kg.

31. A system, kit, or article of manufacture suitable for use in carrying out a method of any one of claim 1-12 or 20-30.

32. A system, kit, or article of manufacture comprising a biomarker of any one of claims 13-19.
